(19) United States
(12) Patent Application Publication
(10) Pub. No.: US 2011/0172106 A1
(43) Pub. Date: Jul. 14, 2011

(54) Detection of Interactions Between Lipid Complexes and Lipid Binding Agents

(75) Inventors: Hugh Willison, Glasgow (GB); Carl Goodyear, Glasgow (GB); Kathryn Brennan, Glasgow (GB); Simon Rinaldi, Glasgow (GB)

(73) Assignee: THE UNIVERSITY COURT OF THE UNIVERSITY OF GLASGOW, Glasgow (GB)

(21) Appl. No.: 13/001,945

(22) PCT Filed: Jul. 3, 2009
(86) PCT No.: PCT/GB2009/001665

§ 371 Date: Mar. 31, 2011

(30) Foreign Application Priority Data
Jul. 3, 2008 (GB) 0812232.7

(51) Int. Cl. C40B 20/02; G01N 33/92; C40B 30/04; C12Q 1/70; C40B 40/04; C40B 50/00; C08F 8/00

(52) U.S. Cl. 506/3; 436/501; 506/9; 435/5; 435/7.1; 506/15; 506/23; 525/326.2

(57) ABSTRACT

The invention relates to materials and methods for detecting interactions between lipid complexes and lipid binding agents. More specifically, the invention provides materials and methods for displaying lipid complexes, particularly those containing two or more different lipid molecules, on a hydrophobic surface so as to mimic their in vivo environment more closely than in other analytical methods. This allows more accurate detection of lipid complexes and even identification of lipid complexes which are not detected by other methods. The invention lends itself particularly well to array or microarray formats.

### FIELD OF THE INVENTION

The present invention relates to materials and methods for detecting interactions between lipid complexes and lipid binding agents.

### BACKGROUND OF THE INVENTION

Within the field of lipidomics, there is an increasing understanding of the molecular processes by which, in the living membrane of cells, different classes of lipid interact closely with each other and with other membrane components, including proteins. Many classes of lipid exist, the major ones being fatty acids, glycerolipids, glycerophospholipids, sterol lipids including cholesterol, and sphingolipids (Wenk, 2005). Within the category of sphingolipids, the headgroups may be glycosylated to form classes of neutral and acidic glycosphingolipids (GSLs). Gangliosides are a class of GSLs containing sialic acid.

Interactions between lipids, especially GSLs, have been shown to be functionally important in cancer cell motility and invasiveness, and in embryogenesis (Regina and Hakomori, 2008). GSLs also act as ligands for many bacterial toxins and for the class of cell signalling molecules known as siglecs (sialic-acid-binding-immunoglobulin-like-lectins) (Schiavo and van der Goot, 2001 and Crocker et al., 2007). Many antibodies bind lipids, both in experimental situations where antibodies have been developed as probes, and in immune and autoimmune states in which anti-lipid antibodies can be detected in the circulation of humans and other species. In the post-infectious inflammatory neuropathy, Guillain Barré syndrome (GBS), anti-GSL antibodies are present. GBS is now the leading cause of acquired paralysis. In these conditions, antibodies which react against particular GSLs, such as gangliosides, can sometimes be detected in patients' sera, but in many cases these cannot be found (Willison, 2005). However, it has recently been shown that while certain patients show reactivity to pairs of GSLs (e.g. ganglioside complexes), antibodies from these patients fail to react with the component species in isolation (Kaida et al., 2004).

At present, the long-established technique of enzyme-linked immunoabsorbant assay (ELISA) is used to evaluate anti-ganglioside antibody activity in patient sera (Willison et al., 1999), both in the research and clinical diagnostic settings. Typically, 10 or so individual target glycolipids are screened on 96-well polystyrene ELISA plates against 100 microlitres of diluted patient serum per ELISA well in this system. In some situations, accessory lipids, such as glycerophospholipids or sterols, have be added in various ratios to improve antibody binding to glycolipids, although this has not been systematically evaluated to the point of being widely incorporated into standard assay methods.

When using the ELISA technique to investigate reactivity of patient sera to GSL complexes, a more limited panel of single GSLs applied in pairs has been used. Even with these reduced numbers, the combinatorial approach using 7 pairs of glycolipids required the production of over 20 different samples, applied manually and in duplicate to separate ELISA wells, for each iteration of the experiment (Kaida et al., 2004 and Kaida et al., 2007). If 10 or more species are used in combination, the number of samples rapidly begins to exceed the capacity of a standard 96-well ELISA plate and consumes so much of the patient serum test reagent that this technique becomes impractical. When glycolipid complexes are formed from more than 2 partners (for example, 10 glycolipids or lipids in clusters of 4), the number of combinations rises rapidly (to 210 in this example) and thus requires a level of miniaturisation that cannot be achieved or practically conducted using standard 96 well ELISA plates.

There are well founded and widely recognised concerns that interactions between lipids (e.g. glycolipids) in an artificial system, such as ELISA, will not necessarily be representative of the interactions that can be observed in the cell membrane in vivo (Willison, 2005). For example, an anti-GM1 antibody may be able to detect GM1 ganglioside immobilised in an ELISA well, but not be able to detect GM1 when it is present in a living cell membrane. Conversely, an antibody may recognise GM1 in a living cell membrane, but not in an ELISA. The relevance of detection in artificial ELISA systems to in vivo biology is thus questionable.

Single GSL dot-blot on polyvinyldifluoride (PVDF), using a manual approach to spot individual species, has previously been described (Chabraoui et al., 1993). Single lipids and glycolipids have also been automatically arrayed onto PVDF membranes and probed with cerebrospinal fluid (CSF) and serum samples from patients with multiple sclerosis (Kanter et al., 2006). Commercially produced nitrocellulose membranes impregnated with single glycolipids are also available (e.g. SphingoStrips™, Molecule Probes, USA).

### SUMMARY OF THE INVENTION

At its most general, the invention relates to methods for detecting interactions between complexes of lipids and lipid binding agents.

In a first aspect, the invention provides a method comprising the steps of:
(i) providing a hydrophobic support displaying a lipid complex;
(ii) contacting the lipid complex with a sample; and
(iii) detecting binding of one or more components of the sample to the lipid complex.

Binding of one or more components of the sample to the lipid complex thus indicates that the sample comprises a lipid binding agent.

This method may be used to detect the presence of a lipid binding agent in a sample. For example, it may be used to determine whether a sample contains a binding agent capable of binding to a specific lipid complex. A sample which is known or suspected to contain an agent capable of binding to one or more lipid complexes may therefore be tested against a plurality of different lipid complexes to determine whether such binding agents are present, and to which complexes they are capable of binding.

The method typically comprises the step of identifying the lipid complex or complexes to which binding occurs.

The plurality of different lipid complexes may be displayed on the same support or on a plurality of supports, depending on the format of the assay. Where the plurality of complexes are displayed on the same support, each complex will typically be displayed at a defined, separate location on the support. Supports carrying a plurality of complexes in this way may be referred to as “arrays”, or “microarrays”, especially when the various locations are arranged in a regular geometric pattern. Thus it may be possible to identify the complex or complexes to which binding occurs by identifying the location at which a positive binding reaction is obtained, and correlating that result with the identity of the complex displayed at that location.

The method may be used to test a biological sample to see whether such lipid binding agents are present. The sample may be a biological fluid, e.g. blood (or a component thereof, such as serum or plasma), cerebrospinal fluid (CSF), saliva, mucous, or urine. The sample may also comprise one or more cells or other biological structures which might contain or comprise a lipid binding agent. Cells may be animal, plant or microbial (e.g. bacterial or fungal) cells. Other structures may include infectious agents such as viruses. Additionally or alternatively the sample may comprise an extract of a cell, virus, etc., or a component isolated therefrom.

The method may thus be used for diagnosis of diseases which are characterised by the presence of lipid binding agents. These diseases include autoimmune diseases (e.g. Guillain Barré syndrome (GBS) and multiple sclerosis) in which affected individuals have antibodies against particular lipid complexes (e.g. glycolipid complexes). They may also include diseases caused by infectious agents which produce lipid binding agents, either on their surface or as secreted molecules, e.g. bacterial toxins such as the cholera, tetanus, shigella and botulinum toxins, and enzymes, such as neuraminidase, which is found on the surface of the influenza virus. Therefore, this method may also be used for the diagnosis of diseases such as cholera, tetanus, shigellosis, botulism and influenza.

The methods of the invention may further be used for assessing the repertoire of binding agents, such as natural antibodies, in normal populations, and thereby relating this to disease susceptibility or protective traits.

The method of the invention may also be used to determine whether a particular test compound is capable of binding to a lipid complex. Thus the method may be used to identify an agent capable of binding to a specific lipid complex, for example by testing a plurality of test compounds for their ability to bind to a lipid complex of choice. The method may comprise selecting a test compound which is capable of binding to the lipid complex.

This type of method may therefore be carried out by screening a library of test compounds against the same lipid complex to see which (if any) of the test compounds are capable of binding to it.

Thus the method may make use of a plurality of supports each carrying the lipid complex, which may be contacted individually with individual test compounds.

Alternatively, the method may make use of a single support carrying the same lipid complex at a plurality of defined, separate locations. In such cases, each test compound may be contacted to an individual and distinct location on the support at which the lipid complex is present. Typically it will be known which test compound was applied to each location. Thus it may be possible to identify those test compound or compounds capable of binding to the complex by identifying the location at which a positive binding reaction is obtained, and correlating that result with the identity of the test compound applied to that location.

In these embodiments of the invention the sample comprises a test compound, which may be a small molecule (e.g. less than 500 Da in molecular weight) or a larger molecule such as an antibody (e.g. a monoclonal antibody), a lectin (for example a siglec or a siglec-Fc fusion protein), or a bacterial toxin (e.g. the cholera, tetanus, shigella or botulinum toxins). Thus, the method may be used to detect binding of the test compound to a lipid complex. As such, this method may be used for the identification of therapeutic or diagnostic agents, such as antibodies, which bind to particular lipid complexes. Such agents may be useful for the diagnosis or treatment of diseases, such as cancer, in which lipid expression is altered (e.g. for the treatment of cancers in which expression of glycolipids is altered, such as melanoma).

In addition, the method may also be used to determine the amount of a lipid binding agent in a sample. Therefore, this method may be used for the diagnosis of diseases in which the levels of lipid binding agents are dysregulated. For example, the anti-metastasis factor CD82, which interacts with the ganglioside complex GM2/GM3, is dysregulated in many types of cancer.

Alternatively the method may be used to investigate the specificity of a test compound which is already known (or suspected) to bind to lipid complexes. Thus the invention provides a method of determining whether a lipid binding agent binds to a particular lipid complex, the method comprising the steps of:
(i) providing a hydrophobic support displaying the lipid complex;
(ii) contacting the lipid complex with said lipid binding agent; and
(iii) detecting binding of said lipid binding agent to the lipid complex.

The method may comprise contacting the lipid binding agent with a plurality of lipid complexes in order to determine which complex or complexes are bound by the agent.

The method will typically comprise the step of identifying the lipid complex or complexes to which binding occurs.

As described above, the plurality of different lipid complexes may be displayed on the same support or on a plurality of supports, depending on the format of the assay. Where the plurality of complexes are displayed on the same support, each complex will typically be displayed at a defined, separate location on the support. It will therefore be possible to identify the complex to which the agent binds by identifying the location at which a positive binding reaction is obtained, and correlating that result with the identity of the complex displayed at that location.

This method may be used to identify lipid complexes bound by known lipid binding agents, including lectins (e.g. siglecs) and antibodies (e.g. serum immunoglobulins and monoclonal antibodies). In particular, if the lipid complex identified by this method is known to be aberrantly expressed in a particular disease, such as a cancer, the method may be used to identify particular lipid binding agents as potential therapeutic or diagnostic agents. Alternatively, if a particular lipid binding agent is known to specifically recognise a particular type of cell or disease state, this method may be used to identify the lipid complex to which it binds, thus identifying that complex as a marker of that cell or disease state.

According to a further aspect of the invention, there may be provided a method of detecting the presence of a lipid complex in a sample, the method comprising the steps of:
(i) displaying the sample on a hydrophobic support;
(ii) contacting the sample with a known lipid binding agent; and
(iii) detecting binding of said lipid binding agent to the sample.

Binding of the known lipid binding agent to the sample thus indicates the presence of a lipid complex in the sample.

This method may be used for the identification of cells known to carry distinctive lipid complexes not found in other cell types, or to possess distinctive quantities of a particular lipid complex (e.g. increased or reduced) compared to other cell types. Thus the method may be used (for example) for diagnosis of a disease in which lipid complexes are aberrantly expressed on the cell surface (e.g. certain types of cancer), or for identification of a pathogen.

The sample may therefore comprise one or more biological cells or an infectious agent such as a virus, or an extract thereof (such as a membrane fraction, e.g. a plasma membrane fraction). Cells may be animal, plant or microbial (e.g. bacterial or fungal) cells. For example the sample may be, or may be derived from, a tissue sample from an individual known or suspected to have a particular disease (e.g. cancer) or to be infected with a particular pathogen (e.g. bacterium, virus or other infectious agent).

It will clearly be possible to test a single, sample for reactivity with a plurality of known lipid binding agents. This can be done using a single support carrying a plurality of lipid complexes at defined separate locations as already described.

To increase throughput, it may also be desirable to analyse a number of different samples (e.g. samples from different individuals, or even different samples from the same individual) on the same support. A single support may therefore be used to test a plurality of samples, each against a plurality of lipid complexes (which may be the same or different for each sample). The skilled person will be capable of designing a suitable format for the support, given the teaching in this specification.

The invention also extends to materials for use in the above-described methods, as well as methods for their production.

Thus according to a further aspect of the invention, there is provided a hydrophobic support displaying a lipid complex. It may display a plurality of lipid complexes at distinct defined locations, which complexes may be same or different as described elsewhere in this specification. This hydrophobic support may be used in the methods of the invention described above. Individual locations or complexes may be separated from adjacent locations or complexes by a barrier which acts to reduce or prevent fluid flow between locations, thus preventing cross-contamination in the course of preparing a support or in performing an assay. The barrier may be a hydrophobic barrier of a wax or other suitable material which resists fluid flow between adjacent locations. Alternatively adjacent locations may be separated by a wall. In such embodiments, each individual location may be surrounded by a wall separating it from adjacent locations, so that each location can be regarded as being (or being located within) an individual well on the support. Thus the walls may be arranged in a grid pattern depending on the configuration of the support and the locations thereon.

According to a further aspect of the invention, there may be provided a method of displaying a lipid complex on a hydrophobic support, the method comprising the step of applying the lipid complex to the hydrophobic support. This step of applying the lipid complex to the hydrophobic support may be automated. Preferably the individual components of the complex are mixed together to allow interaction before they are applied to the support. This further facilitates interactions which more accurately reflect those seen in natural biological environments.

According to a further aspect of the invention, there may be provided a kit for detecting binding of a lipid binding agent to a lipid complex, the kit comprising a hydrophobic support displaying a lipid complex. The hydrophobic support may, for example, display a plurality of lipid complexes. The kit may also include positive and negative control reagents, detection reagents and/or methodology including software for reading, analysing and interpreting the array, as set out in more detail below. This kit may be used in any of the methods of the invention.

The following embodiments relate to any of the aspects of the invention described above.

The hydrophobic support may be made from a material which has an advancing contact angle with respect to water of greater than 60°, greater than 65°, greater than 70°, greater than 75°, greater than 80°, greater than 85°, greater than 90, greater than 95°, greater than 100°, greater than 105°, greater than 110°, or greater than 115°.

The hydrophobic support may be made from a material which has an advancing contact angle with respect to water of greater than 75°, greater than 80°, greater than 85°, or greater than 90°.

Examples of suitable materials for making the hydrophobic support may include polyvinylidene fluoride(PVDF), polytetrafluoroethylene (PTFE)/Teflon', polypropylene, polyethersulphate, polyetherimide (PEI), polyurethane, nylon, cellulose, nitrocellulose or silica. For example, the hydrophobic support may be made from a PVDF slurry, or a silica slurry. The hydrophobic support may be a membrane, which may be formed from any of the materials listed above. A SphingoStrip™ may be suitable.

The lipid complex comprises two or more lipids. Each component may, for example, be a fatty acyl (e.g. fatty acid), glycerolipid, glycerophospholipid, sphingolipid, sterol or prenol.

For example, one or more of the lipids may be cholesterol, sphingomyelin, ceramide or digalactosyl diglyceride.

In some embodiments, as described in more detail below, one or more of the components of the complex may be a glycolipids. The complex may also contain one or more non-glycosylated lipids. Alternatively, the complex may comprise only glycolipids or only non-glycosylated lipids.

For example, the lipid complex may comprise one or more glycolipids (which have a carbohydrate component and a lipid component) and/or one or more glycerophospholipids. Bacterial lipids and glycolipids, such as lipopolysaccharide from Pseudomonas aeruginosa or lipooligosaccharide from Campylobacter jejuni may also be included.

Thus the lipid complex may, for example, comprise two or more glycolipids, a glycolipid and a non-glycosylated lipid (e.g. cholesterol, sphingomyelin or phosphatidylcholine, or a complex of two or more lipids, such as cholesterol and phosphatidylethanolamine), or two or more non-glycosylated lipids, which may include sphingosine or phosphatidyl components. As such, the lipid complexes may be heterodimers, or homodimers. The lipid complex may include two or more, three or more, four or more, five or more, or six or more lipids (e.g. the lipid complex may be a complex of sulphatide, monosialoganglioside, cholesterol and phosphatidylethanolamine). The method may include the step of mixing together two or more of the individual components of the complex, and preferably all of the individual components of the complex, before they are applied to the hydrophobic support. In some embodiments, the glycolipid complex may be displayed on the hydrophobic support in duplicate.

As discussed above, each lipid in the lipid complex may be a glycolipid, for example, a glycosphingolipid, such as a ganglioside. The glycolipid complexes may be heterodimers. For example, they may be heterodimers of any two of the following gangliosides: GM1, GM2, GM3, GD1a, GD1b, GD3, GT1a, GT1b, GD1b, GQ1b and asialo-GM1 (e.g. GM2/GT1b and GM1/GD1a).

The lipid complex may be displayed on the hydrophobic support at a distinct defined location. Furthermore, a plurality of different glycolipid complexes may be displayed on the hydrophobic support, each at a distinct defined location on the hydrophobic support.

For example, the support may carry every possible homodimeric and heterodimeric combination of a given set of monomeric lipids. The set may comprise at least 5, at least 10, at least 15, or more monomeric lipids. Each combination may be displayed at one, two, three or even more locations.

The support may carry one or more replicates of a chosen set of lipid complexes.

Additionally or alternatively, a plurality of locations on the support may each carry the same lipid complex, in order to facilitate screening of a plurality of test compounds (e.g. a library of compounds) for binding activity towards a chosen complex.

On a single support, there may be at least 100, at least 200, at least 500, at least 1000, or even more distinct locations each carrying a lipid complex. A particular advantage of the supports described herein is that the assay format can be significantly miniaturised as compared, for example, to a conventional ELISA format. Thus the number of complexes (or locations) per unit area of the support may be significantly larger than is possible in the ELISA format. For example, a single support may have at least 10, at least 20, at least 50, at least 100, at least 200, at least 300, at least 400, at least 500, or even more distinct locations or complexes per square centimetre. These locations or complexes may be in a grid format and such grids may include, for example, more than 5×5, more than 10×10, more than 20×20, more than 30×30, more than 40×40, or more than 50×50 locations or complexes per square centimetre. Each distinct location may therefore have an area of less than 1.0 mm², less than 0.5 mm², less than 0.2 mm², less than 0.1 mm², less than 0.05 mm², less than 0.02 mm², or less than 0.01 mm². If the lipid complexes are spotted on to the support as substantially circular spots, each spot may, for example, have a diameter of less than 1.0 mm, less than 0.5 mm, less than 0.2 mm, less than 0.1 mm, less than 0.05 mm, less than 0.02 mm, or less than 0.01 mm.

The skilled reader will understand that combinations and variations of these various arrangements are also possible.

Knowing the location of each complex on the support, and/or the location at which any given test compound is applied, permits identification of a lipid complex bound by any particular lipid binding agent, or the test compound capable of binding to any given lipid complex.

The lipid binding agent may be, for example, an antibody (e.g. a monoclonal antibody or serum immunoglobulin), a lectin, which may be mammalian, bacterial or plant lectin, (such as a siglec or siglec-Fc fusion protein), a bacterial toxin (e.g. the cholera or tetanus toxin), or any other suitable protein. Other types of molecule such as carbohydrates or nucleic acids (e.g. aptamers) may also be used, as may small molecules (e.g. of 500 Da or less) which possess or are suspected to possess the capacity to bind lipid complexes. As will already be apparent, the methods of the invention may be used to screen a library of any suitable type of compound for lipid binding ability.

The invention will now be described in detail, by way of example, with reference to the accompanying figures.

### DESCRIPTION OF THE FIGURES

FIG. 1 shows examples of combinatorial GSL grids. FIG. 1A shows an example of a 10×10 combinatorial GSL grid and FIG. 1B shows an example of a 23×23 combinatorial GSL grid. FIG. 1C shows the grid key for the combinatorial arrangement in FIGS. 1B, 2B and 2C.

FIG. 2 shows examples of processed membranes. FIG. 2A shows a 10×10 GSL combinatorial grid that has been probed with a serum from a patient with an inflammatory neuropathy. FIGS. 2B and 2C show 23×23 combinatorial lipid grids. The lipid names have been replaced by numbers, as shown in FIG. 1B, using the grid key shown in FIG. 1C. In FIG. 2B, the grid has been probed with serum from a patient with an undiagnosed neurological disorder. In FIG. 2C, the grid has been probed with serum from a patient with multiple sclerosis.

FIG. 3 shows processed membrane arrays developed on X-ray film. These arrays show ganglioside series GSLs illustrating three alternative patterns of binding. In FIG. 3A, siglec-E was used as the probe; in FIG. 3B, the monoclonal antibody mAb MOG26 was used as the probe; and in FIG. 3C, cholera toxin was used as the probe.

FIG. 4 shows array analysis. (A) Siglec-E intensity data; (B) Siglec E binding to the gangliosides GT1b, GM2 and GT1b/GM2 complex; (C) Comparison of monoclonal antibody mAB DG1 and cholera toxin binding to GM1 series complexes.

FIG. 5 shows the differing responses of anti-GM1 mAbs DG1 and DG2 to complexes of gangliosides containing GM1. (A) Illustrative ELISA plates. The ganglioside complex absorbed to each well is established by combining the row and column headings. Wells labelled with ‘x’ are negative controls (methanol only). (B) Illustrative PVDF glycoarrays. DG1 was used as the primary antibody for the left hand membrane, DG2 on the right. The circles enclose duplicate spots of GM1 alone, hexagons denote GM1/GD1a complex. (C) Quantitative ELISA results from 4 independent experiments. (D) Quantitative results from the PVDF glycoarray (n=3). (E) Comparison of the inhibitory effect of GD1a on GM1 binding for DG1 and DG2. *p=0.02 for two sided T-test of DG1 v DG2. For all graphs DG1 is represented by filled bars, DG2 by open bars.

FIG. 6 shows staining of nerve terminals in living tissue. (A) Merged view of FIGS. 6B and 6C; (B) Staining of nerve terminals with bungarotoxin (BTX); (C) Staining of nerve terminals with cholera toxin (CTB) to identify GM1; (D) Merged view of FIGS. 6E and 6F; (E) Staining of nerve terminals with bungarotoxin (BTX); (F) Staining of nerve terminals with the monoclonal antibody DG2; (G) Merged view of FIGS. 6H and 6I; (H) Staining of nerve terminals with bungarotoxin (BTX); (I) Staining of nerve terminals with the monoclonal antibody DG1.

FIG. 7 shows a comparison of mAb MOG26 and Siglec-E reactivities on ELISA and PVDF glycoarray. (A) PVDF glycoarray probed with MOG26. (B) Results from an ELISA array probed with MOG26. (C) PVDF glycoarray probed with Siglec-E. (D) Results from an ELISA array probed with Siglec-E.

### DETAILED DESCRIPTION OF THE INVENTION

### Lipids

Many lipid components of membranes exist, including fatty acyls (e.g. fatty acids), glycerolipids, glycerophospholipids, sphingolipids, sterols and prenols.

These lipid components include, for example, cholesterol, sphingomyelin, ceramide and digalactosyl diglyceride.

Various of these types of lipid possess head groups which comprise one or more carbohydrate moieties. Such lipids will be referred to herein as glycolipids, whichever category of lipid mentioned above they may belong to. Similarly, lipid types which do not contain a carbohydrate moiety will be referred to as non-glycosylated lipids.

Glycolipids may be of particular interest, and comprise a lipid component and a carbohydrate component. Typically, the carbohydrate component forms a polar head-group and the lipid component forms a lipid tail. In nature, glycolipids occur in diverse membrane environments in most species. In the cell membranes of eukaryotes, the carbohydrate element is associated with phospholipids on the exoplasmic surface of the cell membrane and extends from the phospholipid bilayer into the aqueous environment outside the cell.

Examples of glycolipids include galactolipids, and glycosphingolipids (GSLs), such as cerebrosides, gangliosides, globosides, sulphatides, and glycophosphospingolipids. Glycolipids are generally synthesised from ceramides and sphingosine bases. Bacteria and other organisms also produce a number of well known glycolipids, such as lipopolysaccharide from Pseudomonas aeruginosa and lipooligosaccharide from Campylobacter jejuni.

Gangliosides are the most complex mammalian glycolipids and contain negatively charged oligosaccharides with one or more sialic acid residues. They are highly expressed in nerve cells, but are also present in plasma membrane in all other sites throughout the mammalian system. Specific examples of gangliosides include GM1, GM2, GM3, GD1a, GD1b, GD3, GT1a, GT1b, GD1b, GQ1b and GQ1alpha. Asialo-GM1 is a similar structure to GM1, but does not contain sialic acid.

### Lipid Complexes

A lipid complex comprises two or more lipids physically associated with one another. Thus complexes may comprise one or more of any of the various categories of lipid described above, including glycolipids, fatty acids, glycerolipids, glycerophospholipids, and sterols.

A lipid complex may include two or more, three of more, four or more, five or more, or six or more lipids. Each of the individual components may be from any of the categories described. (For example, a single complex may be a complex of sulphatide, monosialoganglioside, cholesterol and phosphatidylethanolamine.)

In certain embodiments, the complex may comprise at least one glycolipid. Other components of the complex may be non-glycosylated lipids or glycolipids.

Thus a lipid complex may, for example, comprise two or more non-glycosylated lipids (which may include sphingosine or phosphatidyl components), two or more glycolipids (such as gangliosphingolipids, e.g. gangliosides), or it may comprise a glycolipid and a non-glycosylated lipid (e.g. cholesterol, sphingomyelin or phosphatidylcholine.

When two or more lipids form a complex, this complex can contain binding sites for lipid binding agents which cannot be recognised or bound by binding agents applied to the monomers. Similarly, when two or more different lipids (whether or not of the same category) form a complex, this complex can contain binding sites for lipid binding agents which cannot be recognised or bound by binding agents applied to homogeneous complexes of the individual components. Therefore, it is believed that heterologous complexes (i.e. heterodimeric complexes and higher order complexes of two or more different lipids), may possess binding sites for lipid binding agents which are not found on the monomers themselves, or on homogeneous (e.g. homodimeric) complexes of a single lipid. However, the methods of the invention may nevertheless find use with homodimer lipid complexes.

A glycolipid complex may comprise two or more, three or more, four or more, five or more, or six or more glycolipids, such as glycosphingolipids, e.g. gangliosides. However, the glycolipid complex is not limited to a mixture of the same type of molecule. For example, the glycolipid complex may be a mixture of a glycosphingolipid and a ganglioside. These glycolipid complexes may be heterodimers. For example, they may be heterodimers of any two of the following gangliosides: GM1, GM2, GM3, GD1a, GD1b, GD3, GT1a, GT1b, GD1b, GQ1b and asialo-GM1 (e.g. GM2/GT1b and GM1/GD1a).

The individual components of the lipid complex may be mixed before they are used in the methods described herein. This allows interaction of the lipids before they are applied to the hydrophobic support. This facilitates interactions between the lipids which more accurately reflect those seen in vivo.

### Lipid Binding Agents

Lipid binding agents are agents which bind to lipids. Typically, they bind at least in part to a head group on the lipid. This headgroup may include a wide range of chemical modifications, such as inositol, glycerol and phosphate groups. On glycolipids, this head group is or comprises a carbohydrate molecule.

A range of agents may act as lipid binding agents. Examples of such agents which occur naturally include antibodies, lectins (e.g. siglecs) and bacterial toxins (e.g. the cholera, tetanus, shigella or botulinum toxins). The methods of the invention extend to use of, and screening for, agents which do not occur in vivo, such as small molecules capable of binding to particular complexes, monoclonal antibodies, nucleic acids (e.g. aptamers), etc. As already described, the methods of the invention may be used to screen a library of any suitable type of compound for lipid binding ability.

### Samples

As described herein, the methods of the invention may be used to detect the presence of a lipid binding agent in a sample, or to detect the presence of a given lipid complex in a sample. Suitable samples for use in such methods include biological fluids and tissue samples taken from individuals affected by, or suspected of being affected by, particular conditions as well as samples containing, or isolated from other types of organism or infectious agent such as microbial (bacterial or fungal) cells and viruses. Suitable biological fluids include blood (and components thereof including serum and plasma), urine, saliva, mucous and cerebrospinal fluid (CSF). These individuals may be healthy individuals, or may be suspected of having an autoimmune disease, such as Guillain Barré syndrome (GBS), multiple sclerosis, or an infectious disease, such as cholera or influenza.

Tissue samples may include biopsy samples, of normal and/or neoplastic (e.g. cancerous) or inflamed tissue.

In some embodiments, the sample for use in this detection method may include a test compound, such as an antibody (e.g. a monoclonal antibody), a siglec (e.g. a siglec-Fc fusion protein), or a bacterial toxin (e.g. the cholera or tetanus toxin).

### Hydrophobic Supports

Hydrophobic supports displaying lipid complexes are used in the methods of the present invention. The hydrophobic support may be made from a material which has an advancing contact angle with respect to water of greater than 60°, greater than 65°, greater than 70°, greater than 75°, greater than 80°, greater than 85°, greater than 90°, greater than 95°, greater than 100°, greater than 105°, greater than 1100, or greater than 115°.

The hydrophobic support may be made from a material which has an advancing contact angle of greater with respect to water than 75°, greater than 80°, greater than 85°, or greater than 90°.

The advancing contact angle of a material with respect to water can be measured, for example, by depositing a water drop (e.g. having a volume of about 2 μl) on the surface of the material using, for example, a syringe. The advancing contact angle at the interface between the water drop and the material can then be measured using a contact angle meter, such as a contact angle goniometer.

Examples of suitable materials may include polyvinylidene fluoride(PVDF), polytetrafluoroethylene (PTFE)/Teflon™, polypropylene, polyethersulphate, polyetherimide (PEI), polyurethane, nylon, cellulose, nitrocellulose or silica. For example, the hydrophobic support may be made from a PVDF slurry, or a silica slurry. The hydrophobic support may be a membrane, which may be formed from any of the materials listed above. A SphingoStrip™ may be suitable.

The hydrophobic support may itself be supported on a solid substrate, which may be made from any suitable material such as plastics material or glass. In a particularly convenient format, the hydrophobic support is supported on a conventional glass microscope slide (e.g. 6 cm×2 cm). Thus the support may be formed by applying a PVDF slurry or a silica slurry to a glass microscope slide and allowing the slurry to solidify into a membrane.

Without wishing to be bound by any particular theory, it is believed that the hydrophobic lipid components of the lipids tend to associate with, or interact with, the hydrophobic support via hydrophobic interactions (van der Waals interactions). Therefore, binding of lipid complexes to the hydrophobic supports is facilitated by the hydrophobic nature of these supports. Some classes of lipids comprise a polar head group, such as a inositol, glycerol, phosphate or carbohydrate group, in addition to their lipid component. For example, glycolipids comprise a hydrophobic lipid component and a carbohydrate component, which is typically polar and/or charged. Therefore, any polar head group (e.g. a carbohydrate component, such as an oligosaccharide chain) is typically not anchored to the hydrophobic support, and is (to some extent at least) free to move and interact with neighbouring head groups. Interaction between neighbouring head groups (e.g. oligosaccharide chains) is thought to be important for the formation of lipid complexes in a manner that may be representative of the situation found in biological membranes. In particular, this orientation of the lipids (e.g. glycolipids) may also improve accessibility of their head groups (e.g. carbohydrate components) for binding to lipid binding agents, which may assist in the binding required for the methods of the invention. In particular, glycolipid binding agents usually bind to the carbohydrate components of glycolipid complexes, although the binding site may in some cases extend onto the lipid component of the complex.

The presence of a non-glycosylated lipid in a glycolipid complex (to form a non-glycosylated lipid lipid/glycolipid complex) may further aid the stabilisation of the complex in a manner that permits binding of a binding agent that may not be evident using other methods. Complexes made up entirely of non-glycoslyated lipids (e.g. non-glycosylated lipid dimers) may also be displayed on hydrophobic supports and may this may permit binding of lipid-binding agents that may not be evident using other methods.

Therefore, the orientation of lipid complexes (e.g. glycolipid complexes) bound to hydrophobic supports, as well as the interactions between each lipid in the complex, is thought to be more representative of the in vivo situation than when other test systems (such as ELISA) are used.

The application of lipid complexes to the hydrophobic support may be automated. Each lipid complex is preferably applied to the hydrophobic support in duplicate (e.g. as duplicate spots). The lipid complexes may be spotted onto the hydrophobic support, preferably at a distinct, defined location. This allows identification of a lipid complex bound by a lipid binding agent through correlation of a positive binding reaction with a particular location on the hydrophobic support. Preferably two or more of the individual lipid components (and preferably all of the components) are mixed together and allowed to interact with one another before they are applied to the support.

The lipid complexes may be applied to the hydrophobic support in a particular pattern or configuration. For example, a plurality of lipid complexes may be applied to the hydrophobic support in a regular array (e.g. a grid).

Suitable grid matrices include, for example, 10×10 (rows×columns), 20×20, 23×23, 30×30, 40×40, 50×50, 100×100 and 200×200 grids. Preferably, the grid matrices are up to 1000×1000, up to 500×500, up to 200×200, up to 100×100, or up to 50×50 in size. The hydrophobic support may, for example, be supported on a solid substrate, such as glass. For example it may be attached to a conventional glass microscope slide (e.g. 20×60 mm). Grids can, however, be much larger than this (e.g. 200×200 mm) for more complex applications, especially when complexes comprising more than two glycolipids are used.

A single support may comprise at least 100, at least 200, at least 500, at least 1000 or even more distinct locations, each carrying a lipid complex. A particular advantage of the assay format of the invention is that it can be significantly miniaturised as compared, e.g. to a conventional ELISA format. Thus a much higher density of complexes may be applied and tested per unit area of the support. For example, a single support may have at least 10, at least 20, at least 50, at least 100, at least 200, at least 300, at least 400, at least 500 or even more distinct locations or complexes per square centimetre. These locations or complexes may be in a grid format and such grids may include, for example, more than 5×5, more than 10×10, more than 20×20, more than 30×30, more than 40×40, or more than 50×50 locations or complexes per square centimetre. Each distinct location may therefore, have an area of less than 1.0 mm², less than 0.5 mm², less than 0.2 mm², less than 0.1 mm², less than 0.05 mm², less than 0.02 mm², or less than 0.01 mm². If the lipid complexes are spotted on to the support as substantially circular spots, each spot may, for example, have a diameter of less than 1.0 mm, less than 0.5 mm, less than 0.2 mm, less than 0.1 mm, less than 0.05 mm, less than 0.02 mm, or less than 0.01 mm.

### Detection Methods

Disclosed herein is a method comprising the steps of: (i) providing a hydrophobic support displaying a lipid complex; (ii) contacting the lipid complex with a sample; and (iii) detecting binding of one or more components of the sample to the lipid complex.

Binding of one or more components of the sample to the lipid complex thus indicates that the sample comprises a lipid binding agent.

The method may also include one or more of the following steps.

(a) The method may include the step of mixing two or more individual components of the lipid complex (and preferably all of the components of the complex) before they are applied to the hydrophobic support. This allows interaction between the components before they are applied to the hydrophobic support, thus facilitating interactions which more accurately reflect those seen in vivo.

(b) The method may include the step of applying a blocking solution containing a component which does not have substantial lipid binding ability (e.g. a protein, such as bovine serum albumin) to the hydrophobic support before contacting the support with the sample. This can help to prevent non-specific interaction between lipid binding substances or other substances in the sample and the support and/or lipid complexes displayed thereon.

(c) The method may include the step of applying the lipid complex to the hydrophobic support, or the method may make used of a hydrophobic support onto which a lipid complex has already been applied. The hydrophobic support displaying the lipid complex is then contacted with a sample to be screened for the presence of lipid binding agents.

(d) The method may also include the step of identifying the location of the lipid complex bound by a lipid binding agent and correlating this location with the identity of the lipid complex. This permits identification of the lipid complex bound by the lipid binding agent, especially if the lipid complex is displayed on the hydrophobic support at a defined distinct location.

Various methods can be used to detect such binding. For example, a labelled antibody (e.g. an antibody linked to horse radish peroxidise (HRP)) can be used to detect agents bound to the lipid complexes. Binding of this labelled antibody can then be detected using, for example, a chemiluminescence reaction. Alternatively, components of the sample (e.g. siglec-Fc fusion proteins or bacterial toxin conjugates) can be directly labelled, e.g. by conjugation to HRP, or to an HRP-liked anti-Fc antibody, in the case of siglec-Fc fusion proteins. Binding of these agents to the lipid complexes displayed on the hydrophobic support can then be detected using, for example, a chemiluminescent reaction. Alternatively, binding can be detected using chemifluorescence reactions. Chemifluorescence can, for example, be detected using a phosphoimager. In addition, fluorescently labelled secondary antibodies may be used to detect lipid binding agents bound to the lipid complexes displayed on the hydrophobic support.

This detection method may be used to detect the presence of lipid binding agents (such as antibodies) in a sample taken from a patient (e.g. a serum sample or a sample of CSF). Therefore, this method can be used for the diagnosis of a variety of autoimmune diseases, such as Guillain-Barré syndrome (GBS), in which auto-antibodies against particular glycosphingolipids (typically gangliosides) are produced, or multiple sclerosis.

This detection method can also be used for the detection of diseases caused by infectious agents which produce lipid binding agents, either on their surface or as secreted molecules, e.g. bacterial toxins such as the cholera, tetanus, shigella and botulinum toxins and enzymes, such as neuraminidase (which is found on the surface of the influenza virus). Therefore, this method may also be used for the diagnosis of diseases, such as cholera, tetanus, shigellosis, botulism and influenza, which are caused by infectious agents.

For example, cholera toxin binds to GM1 series complexes and tetanus toxin binds to ganglioside complexes including GD3/GM2 and GD3/GD1a, as well as GD1b and GT1b series complexes. Therefore, a method for the detection or diagnosis of cholera may involve the use of a hydrophobic support which displays one or more GM1 ganglioside complexes. Similarly, a kit for the detection or diagnosis of cholera may comprise a hydrophobic support which displays one or more GM1 ganglioside complexes. A method for the detection or diagnosis of tetanus may involve the use of a hydrophobic support which displays one or more GD3/GM2, GD3/GD1a, GD1b and/or GT1b ganglioside complexes. Similarly, a kit for the detection or diagnosis of tetanus may comprise a hydrophobic support which displays one or more GD3/GM2, GD3/GD1a, GD1b and/or GT1b ganglioside complexes.

These diagnostic methods can include the step of obtaining a sample from a patient (such as blood sample (e.g. a serum sample), or a urine, saliva, mucous or CSF sample), or can be carried out using a sample that has already been obtained from a patient. This sample is then used to contact a hydrophobic support comprising displaying a lipid complex.

Binding of one or more antibodies in the sample to be screened to one or more of the lipid complexes displayed on the hydrophobic support may indicate that the patient has a disease, such as an autoimmune disease, e.g. GBS or multiple sclerosis. The presence of a lipid binding agent in the sample may also indicate the susceptibility of an individual to developing a disease or provide an indication of the level of immunity present against an infectious agent.

Such binding of antibodies to the lipid complexes arrayed on the hydrophobic support can be detected using various methods, as described in the “Methods of screening” section above. For example, a labelled antibody (e.g. an antibody linked to horse radish peroxidise (HRP)) can be used to detect antibodies bound to the lipid complexes. Binding of antibodies to the lipid complexes arrayed on the hydrophobic support can then be detected, for example using a chemiluminescent reaction. Alternatively, binding can be detected using chemifluorescence reactions using, for example, a phosphoimager. In addition, fluorescently labelled secondary antibodies may be used to detect antibodies bound to the lipid complexes arrayed on the hydrophobic support.

This detection method may also be used to detect binding of a test compound in a sample to a lipid complex. The test compound may be an antibody (e.g. a monoclonal antibody), lectin (such as a siglec, e.g. a siglec-Fc fusion protein), a bacterial toxin (e.g. the tetanus or cholera toxins), or anylectin, protein or nucleic acid. Thus, the method may be used to detect binding of the test compound to a lipid complex. As such, this method may be used for the identification of therapeutic agents, such as therapeutic antibodies, which bind to particular lipid complexes. Therapeutic agents identified by this method may be useful for the treatment of diseases, such as cancer, in which lipid expression is altered (e.g. for the treatment of cancers in which expression of glycolipids is altered). For example, melanoma cells display altered ganglioside profiles (Lloyd et al., 1982).

In addition, the method may also be used to determine the amount of a particular lipid binding agent in a sample. Therefore, this method may be used for the diagnosis of diseases in which the levels of lipid binding agents are dysregulated. For example, the anti-metastasis factor CD82, which interacts with the ganglioside complex GM2/GM3, is dysregulated in many cancers (Regina and Hakamori, 2008).

The invention also relates to methods of determining whether a known lipid binding agent binds to a particular lipid complex, the method comprising the steps of: (i) providing a hydrophobic support displaying the lipid complex; (ii) contacting the lipid complex with said lipid binding agent; and (iii) detecting binding of said lipid binding agent to the lipid complex.

Binding of said lipid binding agent to the lipid complex thus indicates that the known lipid binding agent binds the lipid complex.

This method may be used to identify lipid complexes bound by known lipid binding agents (such as siglecs (e.g. lectins) and monoclonal antibodies). In particular, if the lipid complex identified by this method is known to be aberrantly expressed in a particular disease, such as cancer (e.g. a melanoma), the method may be used to identify known lipid binding agents as potential therapeutic agents.

As described above, this method may include the step of mixing the individual components of the lipid complex before they are applied to the hydrophobic support and/or the step of applying a blocking solution to the hydrophobic support before contacting the support with the sample.

This method may also include the step of applying the lipid complex to the hydrophobic support, or the method may make use of a hydrophobic support onto which a lipid complex has already been applied. The hydrophobic support displaying the lipid complex is then contacted with a sample to be screened for the presence of lipid binding agents.

The method may also include the step of identifying the location of the lipid complex bound by a lipid binding agent and correlating this location with the identity of the lipid complex. This permits identification of the lipid complex bound by the lipid binding agent, especially if the lipid complex is displayed on the hydrophobic support at a defined distinct location.

As described above, various methods can be used to detect such binding of the known lipid binding agent to the lipid complex. For example, a labelled antibody (e.g. an antibody linked to horse radish peroxidise (HRP)) can be used to detect agents bound to the glycolipid complexes. Binding of this labelled antibody can then be detected using, for example, a chemiluminescence reaction. Alternatively, components of the sample (e.g. siglec-Fc fusion proteins or bacterial toxin conjugates) can be directly labelled, e.g. by conjugation to HRP, or to an HRP-liked anti-Fc antibody, in the case of siglec-Fc fusion proteins. Binding of these agents to the lipid complexes displayed on the hydrophobic support can then be detected using, for example, a chemiluminescent reaction. Alternatively, binding can be detected using chemifluorescence reactions. Chemifluorescence can, for example, be detected using a phosphoimager. In addition, fluorescently labelled secondary antibodies may be used to detect lipid binding agents bound to the lipid complexes displayed on the hydrophobic support.

The invention also relates to a method for detecting the presence of a lipid complex in a sample, the method comprising the steps of: (i) displaying the sample on a hydrophobic support; (ii) contacting the sample with a known lipid binding agent; and (iii) detecting binding of said lipid binding agent to the sample.

Binding of the known lipid binding agent to the sample thus indicates the presence of a lipid complex in a sample.

This method may be used for the diagnosis of a disease in which lipids are aberrantly expressed on the cell surface (e.g. certain types of cancer, such as melanoma). For example, the sample may be a tissue sample from a patient, or a biopsy sample.

This method may include the step of obtaining a sample from a patient (e.g. a tissue sample, or a biopsy sample), or can be carried out using a sample that has already been obtained from a patient. This sample is then applied to a hydrophobic support, so that the any lipid complexes present in the sample are displayed on the support.

As described above, various methods can be used to detect such binding of the known lipid binding agent to the lipid complex. For example, a labelled antibody (e.g. an antibody linked to horse radish peroxidise (HRP)) can be used to detect agents bound to the lipid complexes. Binding of this labelled antibody can then be detected using, for example, a chemiluminescence reaction. Alternatively, components of the sample (e.g. siglec-Fc fusion proteins or bacterial toxin conjugates) can be directly labelled, e.g. by conjugation to HRP, or to an HRP-liked anti-Fc antibody, in the case of siglec-Fc fusion proteins. Binding of these agents to the lipid complexes displayed on the hydrophobic support can then be detected using, for example, a chemiluminescent reaction. Alternatively, binding can be detected using chemifluorescence reactions. Chemifluorescence can, for example, be detected using a phosphoimager. In addition, fluorescently labelled secondary antibodies may be used to detect lipid binding agents bound to the lipid complexes displayed on the hydrophobic support.

### Autoimmune Diseases

Several autoimmune diseases are caused, at least partially, by the production of auto-antibodies against particular lipids. For example, antibodies in sera from patients with the post-infectious inflammatory neuropathy, Guillain Barré syndrome (GBS), react against particular glycosphingolipids (typically gangliosides). Other autoimmune diseases in which antibodies against lipids, such as glycosphingolipids, are produced, include multiple sclerosis. In addition, antibodies against phospholipids, such as cardiolipin, are present in the circulation of patients with inflammatory vascular diseases and antibodies binding to oxidised phosphorylcholine (PC)-containing phospholipids are involved in immune defense against microbial infections and may also be involved in binding to self lipid components and contributing to atherosclerosis. Therefore, such diseases can be diagnosed using the methods of the invention.

### Kits

The present invention also relates to kits which comprise a hydrophobic support displaying a lipid complex. For example, the hydrophobic support may be pre-printed with a plurality of lipid complexes. The lipids are preferably glycolipids, such as glycosphingolipids (e.g. gangliosides).

The hydrophobic support may be made from a material which has an advancing contact angle with respect to water of greater than 60°, greater than 65°, greater than 70°, greater than 75°, greater than 80°, greater than 85°, greater than 90°, greater than 95°, greater than 100°, greater than 105°, greater than 110°, or greater than 115°.

The hydrophobic support may be made from a material which has an advancing contact angle with respect to water of greater than 75°, greater than 80°, greater than 85°, or greater than 90°.

Examples of suitable materials may include polyvinylidene fluoride(PVDF), polytetrafluoroethylene (PTFE)/Teflon', polypropylene, polyethersulphate, polyetherimide (PEI), polyurethane, nylon, cellulose, nitrocellulose or silica. For example, the hydrophobic support may be made from a PVDF slurry, or a silica slurry. The hydrophobic support may be a membrane, which may be formed from any of the materials listed above. A SphingoStrip™ may be suitable.

Alternatively the kit may contain the components required for a user to synthesise their own custom array of lipid complexes and to carry out a method of the invention. For example, the kit may comprise a support and a panel of individual lipids (e.g. 5 or more, 10 or more, or 20 or more individual lipids). The user may then combine the panel of lipids with one another, or with other lipids of their choice, and apply them to the support to create their own array of lipid complexes for use in a method of the invention. Individual locations on the support for application of lipid complexes may be pre-printed or otherwise pre-defined.

These kits can be used in the detection methods and diagnostic methods described above. For example, they may be used for the diagnosis of autoimmune diseases, such as GBS and multiple sclerosis, or infectious diseases.

The kits may also include a positive control, i.e. a lipid binding agent which is known to bind to at least one of the complexes present on the membrane. For example, it may be an antibody which binds to a glycolipid complex. The kit may also include a negative control, i.e. a substance which is known not to bind to any of the complexes on the membrane. Where the user prepares their own array, alternative forms of negative control may be provided for application to the support, which will not provide a positive result for any lipid binding agent (e.g. methanol). The kits may also include a labelled secondary antibody (such as an HRP-linked antibody) and/or a detection reagent to allow binding of agents to the lipid complexes to be detected, e.g. by chemiluminescence.

Advantages Associated with Using Hydrophobic Supports to Display Lipids

There are several advantages associated with using hydrophobic supports (such as PVDF membranes) to display lipids (e.g. glycolipids) for detecting lipid binding proteins, rather than known techniques, such as thin layer chromatography or ELISA.

Firstly, an automated sampler can be used to allow multiple different combinations of lipids, e.g. glycolipids, to be spotted on to the hydrophobic support in a highly efficient and stereotyped manner. In contrast, preparing a large number of complexes on ELISA plates is much slower, as well as being technically arduous. In view of the long time taken to prepare ELISA plates with large numbers of complexes, use of ELISA-based methods is liable to generate variation. Therefore, applying lipid complexes to hydrophobic supports makes high throughput screening possible.

Furthermore, as printed hydrophobic supports (e.g. PVDF membranes) may be small (typically about 20×25 mm), only small volumes of test solution (e.g. 250 μl) are required for each 10×10 grid. Using ELISA, 10 ml of solution would be required for testing against the same range of complexes (when using 100 μl/well), which represents a forty-fold reduction in the amount of test solution required. This miniaturisation of the method means that only a small volume of sample, e.g. serum or other biological fluid, needs to be tested for reactivity with a plurality of known lipid binding agents. This is important when testing samples which have a limited availability (e.g. serum samples from patients).

Lipids (e.g. glycolipids) bind to hydrophobic supports (e.g. PVDF membranes) via a hydrophobic interaction with their lipid components (e.g. lipid tail). Therefore, lipids are displayed on hydrophobic supports (e.g. PVDF membranes) in a way that may be more similar to their in vivo orientation. As the head group component (e.g. the carbohydrate component in glycolipids) is not anchored, interaction with neighbouring head group components, which is crucial to the formation of lipid (e.g. glycolipid) complexes, is permitted. In ELISA, depending on the type and composition of the microtitre plate used, lipids (e.g. glycolipids) may bind to the ELISA plate through electrostatic interactions with their head-groups. Therefore, lipid may be displayed differently on ELISA plates in comparison with PVDF or other hydrophobic membranes.

As a consequence of the orientation of lipids (e.g. glycolipids) when displayed (e.g. as an array) on a hydrophobic support, such as a PVDF membrane, the combinatorial glycoarray technique of the present invention has the potential to reflect a different pattern of carbohydrate-carbohydrate and other head group interactions in comparison with ELISA techniques. This pattern of binding to PVDF may in some circumstances better reflect the topographical organisation of lipids (e.g. glycolipids) occurring in living hydrophobic supports (e.g. cell membranes) more accurately than known techniques, such as ELISA. In support of this, experimental data described herein confirm that reactivities of certain glycolipid binding antibodies seen on PVDF are not always consistent with those detected on ELISA, particularly with respect to anti-complex activity.

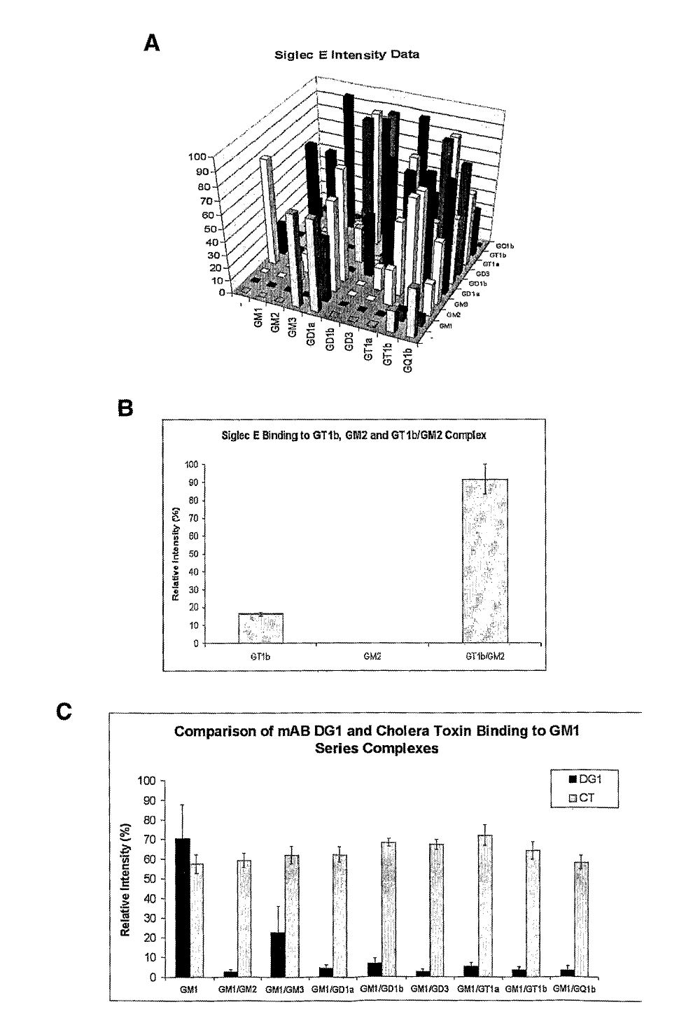

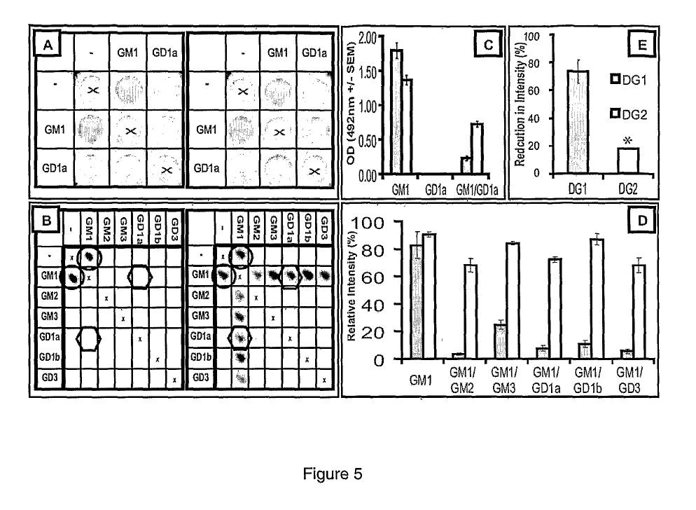

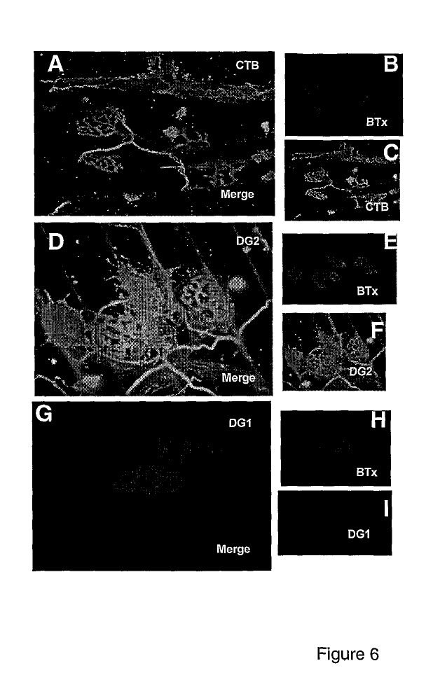

For example, as shown in FIG. 5, a monoclonal antibody (DG1) previously generated by the present inventors reacts significantly with a mixture of GM1/GD1a on ELISA, but not at all with the same complex on PVDF. It also fails to bind at all to living tissue in which GM1/GSL complexes are thought to form (see FIG. 6). In contrast, the antibody DG2 binds GM1 in complex with GD1a and in complex with other GSLs in both ELISA and PVDF glycoarrays (showing that both methods are functioning well in this controlled experiment). DG2 is also able to bind GM1 in living tissue, as shown in FIG. 6. Thus, the PVDF array is able to identify an antibody (DG1), which does not bind GM1 in tissue, as being unable to bind to GM1 complexes, whereas the ELISA is not able to discriminate this as effectively.

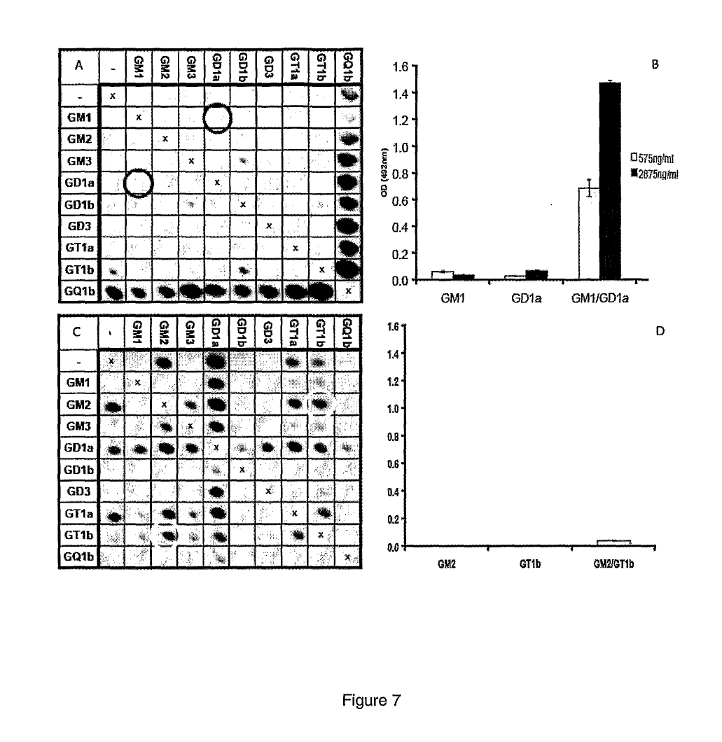

Another example demonstrating the inconsistency of results obtained using ELISA and PVDF is shown in FIG. 7. In this example, the monoclonal antibody MOG26 binds a GM1/GD1a complex on ELISA, but not on PVDF. Consistent with the results obtained using PVDF, MOG26 binds live tissue in transgenic mice which express complexes of GM1/GD1a (data not shown).

These examples strongly suggest that using PVDF-based methods to display lipid complexes (e.g. glycolipid complexes) is likely to be more representative of the in vivo situation than using ELISA-based assays.

### EXAMPLES

### Materials and Methods

Single gangliosides, GSLs and lipids were purchased. GM1, asialo-GM1, GM2, GM3, GM4, GD1a, GD1b, GD3, GT1b, sulphatide, galactocerebroside, sphingomylein, cholesterol, ceramide, and digalactosyl diglyceride were obtained form Sigma, UK. GT1a and GQ1b were obtained from Accurate Chemical and Scientific, USA, and GD2 from Calbiochem, USA. Phosphotidylcholine, phosphotidylserine, phosphotidylethanolamine, phosphatidyl-inisitol-4-phosphate and glycero-3-phosphocoline were obtained from Avanti Polar Lipids, USA.

Stock solutions of each of the above were prepared in a 50:50 (v/v) chloroform:methanol mixture, at 1 to 10 mg/ml. Working solutions were made by further dilution in methanol to 0.1 mg/ml. For single samples, 200 μl of the working solution was added to a 300 μl capacity micro-sampling vial (Chromacol, UK). To create complexes, 100 μl of each constituent GSL was added to a vial. Vials were sealed using caps with a rubber insert (Chromacol, UK), allowing puncture by the autosampler needle. All samples were then sonicated for 3 minutes prior to use.

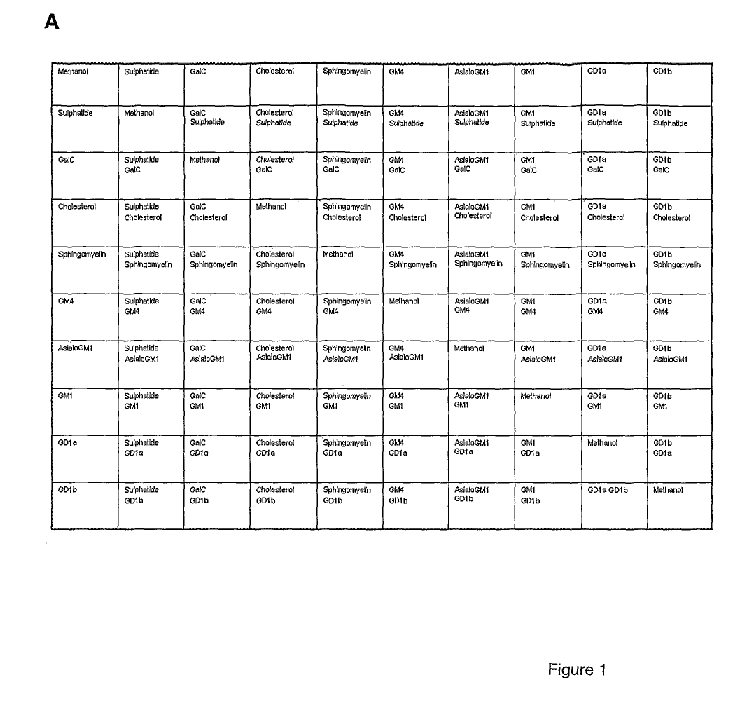

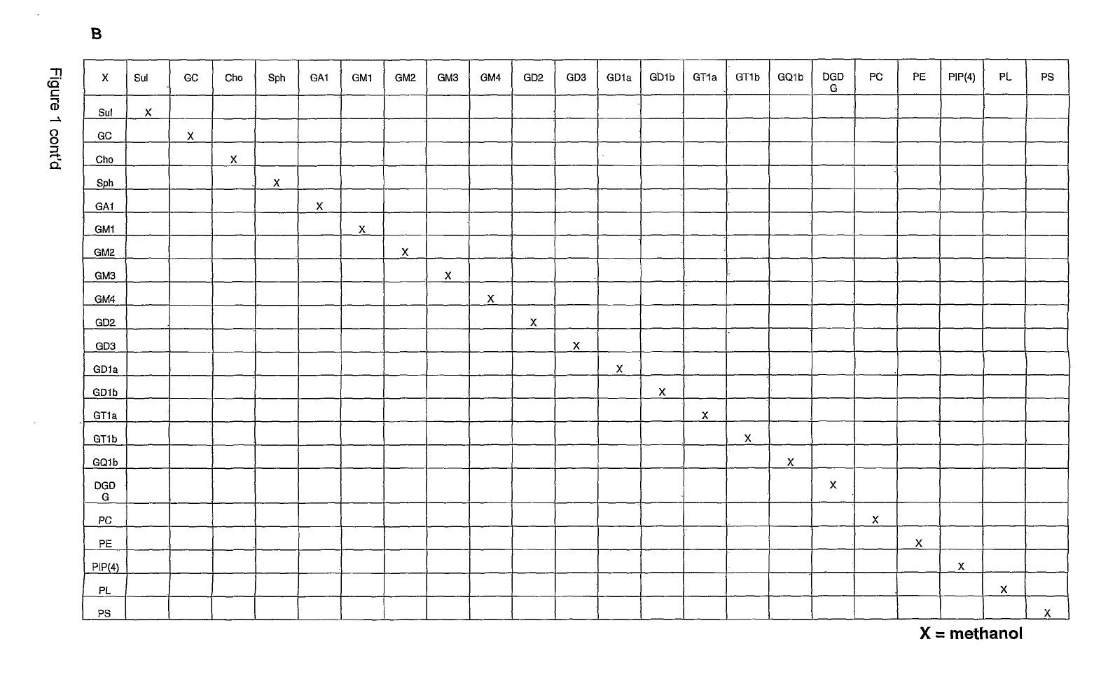

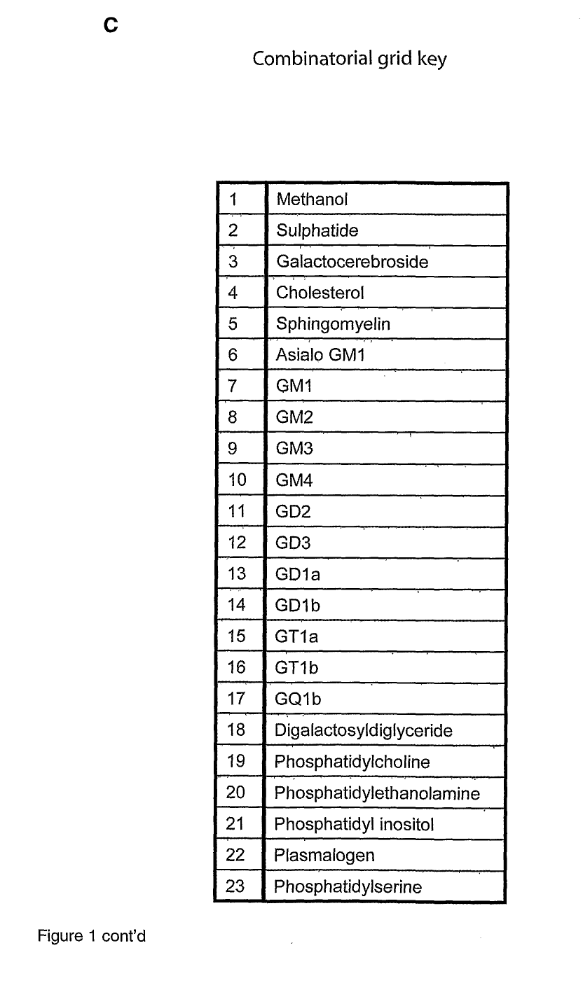

Sheets of PVDF membrane (Sigma, UK) were cut into 20×25 mm squares using a scalpel. These were then affixed 12 mm from the left hand edge of a plain glass slide (VWR International, UK) using UHU glue (UHU GmbH, Germany), and allowed to air dry for 10 minutes. A metal grid was used to hold 12 slides in predefined and consistent positions on the application plate of a Camag Automatic TLC Sampler 4 (Camag, Switzerland). The winCATS planer chromatography management software (Camag, Switzerland) was used to write programs which result in the application of duplicate spots of 0.1 μl of 100 μl/ml ganglioside or ganglioside complex over a predefined 0.4 μm² area. An example of a 10×10 grid is shown in FIG. 1A. Larger grids of 23×23 spots have also been produced, spread over 2 separate slides. Printed hydrophobic supports were outlined with a hydrophobic barrier pen (Vector Laboratories, UK) and allowed to air dry for 20 minutes. They were then stored overnight at 4° C. before use.

Membranes were blocked in at least 100 ml/cm² of 2% bovine serum albumin/phosphate buffer saline (BSA/PBS) for 1 hr at 4° C. Serum samples, CSF, monoclonal antibodies, siglec-Fc fusion proteins (preconjugated to horse radish peroxidase (HRP) linked anti-Fc antibody), or HRP-bacterial toxin conjugates were diluted in 1%; BSA/PBS. 250 μl of this diluted sample was then applied to a pre-printed membrane and incubated at 4° C. After 1 hr, the sample was tipped from the membrane and the slides were briefly placed back in the 2% BSA/PBS blocking solution. Probes requiring a secondary antibody underwent a primary wash phase. These membranes were transferred to at least 500 ml/cm² of 1% BSA/PBS for 15 minutes of washing on a shaker set at 100 rpm. This process was repeated once. These membranes were tapped dry, 250 μl of the appropriate HRP linked secondary antibody was applied (diluted in 1% BSA/PBS), and incubated for 30 m at 4° C. All membranes then entered a wash phase. For probes not requiring a secondary antibody (siglecs and HRP-conjugated bacterial toxins), this immediately followed the primary incubation.

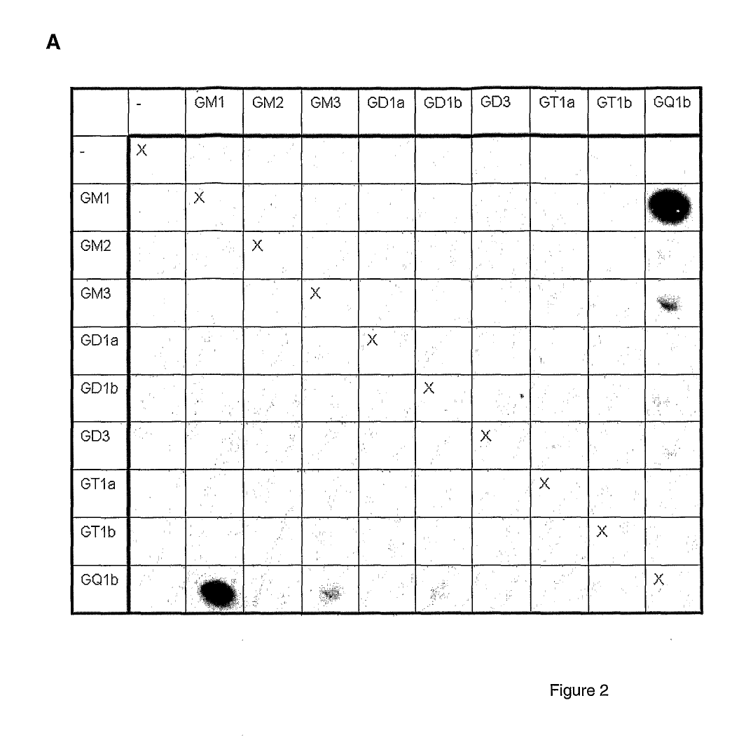

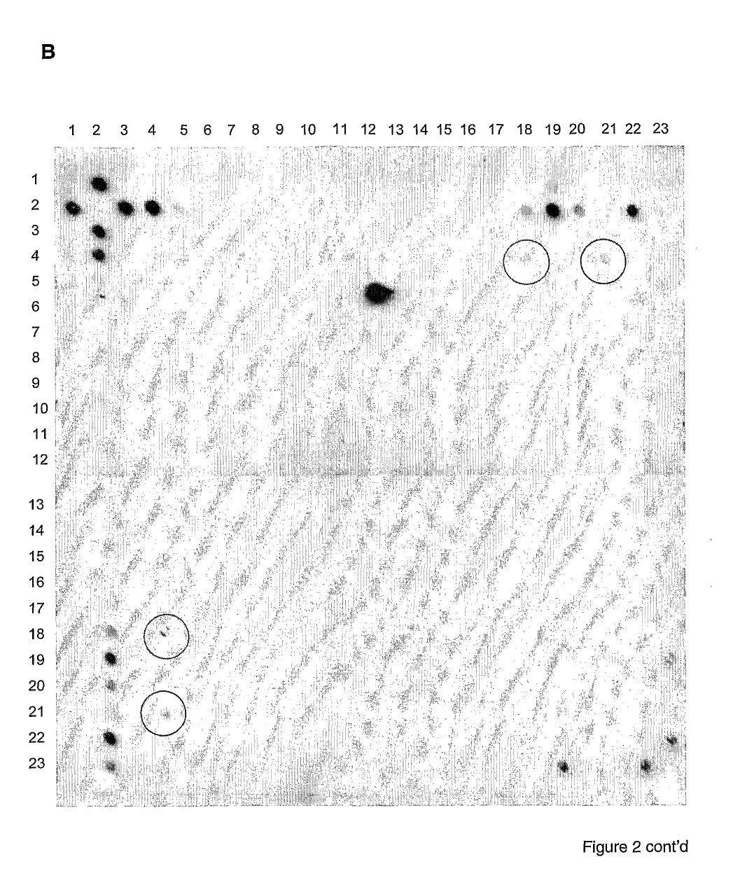

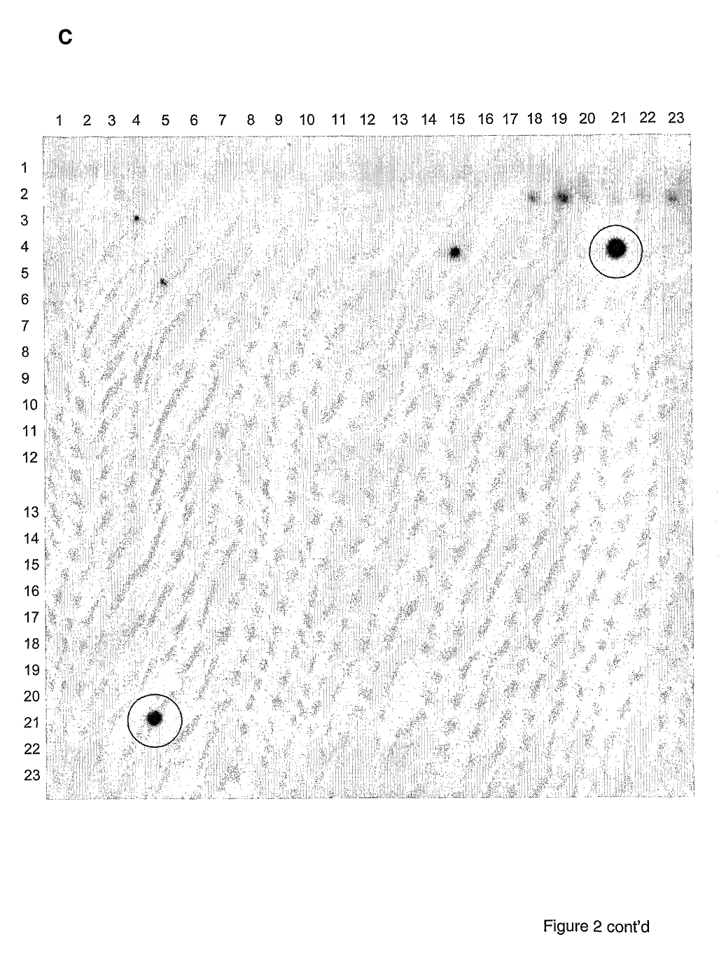

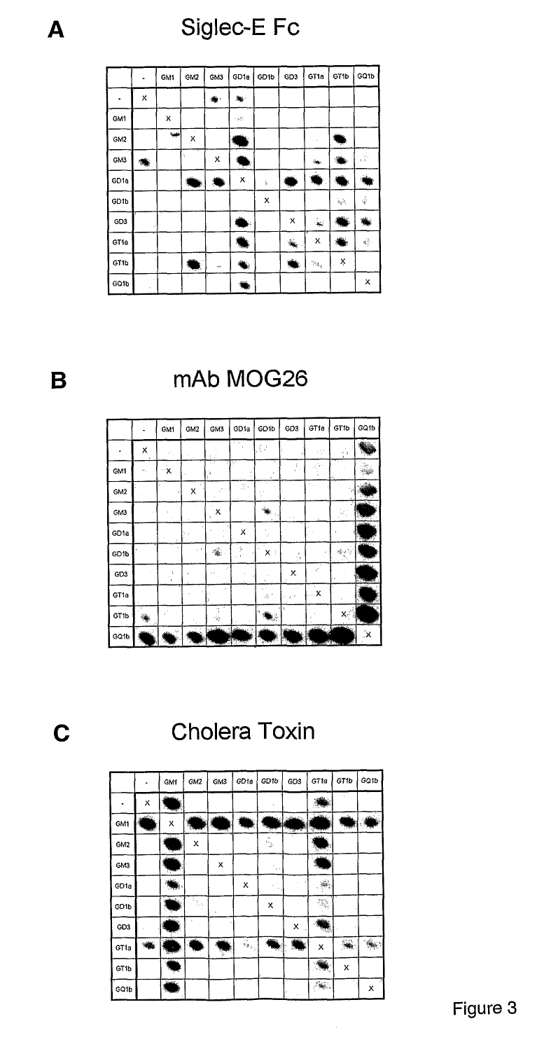

This wash phase consisted of two changes of 1% BSA and three changes of PBS, again each of at least 500 ml/cm². BSA washes were of 30 m duration, PBS for 5 m, both on a shaker set at 100 rpm. Slides were then briefly dipped in two changes of distilled water (500 ml/cm²). A chemiluminescent detection reaction was then performed using ECL plus (Amersham/GE Healthcare, UK), made up according to manufacturer's instructions. 450 μl of this detection solution was then applied to the membranes and left for 3 minutes at room temperature. The solution was tipped from the membranes and signal was detected on radiographic film. Exposure time was initially 15 s; subsequent exposures were adjusted on the basis of this first result. Films were digitised by flatbed scanning and the images analysed and quantified by the array analysis component of ImageQuant TL software (Amersham Biosciences, UK). Examples of processed membranes are shown in FIGS. 2 and 3, as described in detail elsewhere in Examples 2 and 3 below. An example of array analysis is shown in FIG. 4 and described elsewhere.

### Example 1

### Combinatorial GSL/Lipid Grids

Examples of combinatorial GSL/lipid grids are shown in FIGS. 1A and 1B, which show 10×10 and 23×23 grids, respectively. A line of methanol as negative control runs diagonally across the membrane from top left to bottom right corners. This acts as a line of symmetry for duplicate spots within the membrane. The first row and first column contain single species. Other spots are complexes of two GSLs, and consist of the single glycolipid spotted at the extreme left of the row combined with the glycolipid at the top of the column.

### Example 2

### Processed Combinatorial Lipid Grids

A 10×10 GSL combinatorial grid was probed with serum from a patient with an inflammatory neuropathy as the primary probe, followed by an anti-human IgG-HRP linked secondary antibody and then a development step. The prominent positive spot corresponds to the ganglioside complex GM1/GQ1b (see FIG. 2A).

FIG. 2B shows a 23×23 combinatorial lipid grid in which serum from a patient with an undiagnosed neurological disorder was used as the primary probe, followed by with anti-human IgG-HRP linked secondary antibody and then a development step. Sulphatide is spotted in Row 2 and Column 2 and is bound by IgG antibody in this serum when on its own (spot 1,2 and spot 2,1) and when in combination with other lipids (e.g. Spot 2,3; 2,4; 2,19 and 2, 22; in corresponding rows and columns). It should be noted that the combination of sulphatide when complexed with glycolipids spotted at positions 5 through 17 creates an inhibitory interaction that prevents the anti-sulphatide IgG from binding sulphatide. The circled spots show binding to complexes of lipid pair comprising digalactosyl diglyceride/cholesterol and phosphatidyl inositol/cholesterol. Note that neither of these 3 lipids is bound when spotted on its own (i.e. 1,4; 1,18; and 1,21 positions in corresponding rows and columns are negative). The prominent black signal circa position 6,12 is a technical artefact.

FIG. 2C shows a 23×23 combinatorial lipid grid probed with serum from a patient with multiple sclerosis. The prominent positive spot corresponds to the complex of phosphatidyl inositol/cholesterol and is symmetrically present at 4,21. The serum does not react with either phosphatidyl inositol (spot or cholesterol alone (i.e. positions 1,21 and 1,4 are negative). The black signals circa positions 3,4; 4,15 and 5,5 are technical artefacts.

### Example 3

### Alternative Patterns of Ganglioside Binding

In FIG. 3A, a complicated pattern of binding is demonstrated for siglec-E. Some ganglioside pairings attenuate signals obtained with either ganglioside alone and some enhance the signal (e.g. the GM3 signal is attenuated by GM1 and enhanced by GD1a). Intensity data is plotted in FIGS. 4A and 4B. In FIG. 3B, the monoclonal mouse anti-GQ1b antibody mAb MOG26 is shown to bind GQ1b on its own and in the presence of other GSLs. It also binds a combination of GD1b and GM3 whilst binding negligibly to either ganglioside alone. In FIG. 3C, cholera toxin is shown to bind well to GM1 and GT1a, either alone or in combination with other GSLs. GD1a creates a relatively inhibitory environment for cholera toxin binding, suppressing the binding intensity with both GM1 and GT1a.

### Example 4

### Array Analysis

Processed array grids are analysed using ImageQuant software (Amersham Biosciences), which produces a large amount of intensity data (see FIG. 4A). This, along with pictorial representation, is used to identify ganglioside pairs of interest for further evaluation (see FIG. 4B).

Alternatively, binding to a particular ganglioside series of complexes can be compared for different binding agents. For example, binding of anti-GM1 mAb DG1 is inhibited in the presence of any other paired species, whereas cholera toxin is able to bind regardless of the presence or absence of a second ganglioside (see FIG. 4C).

### Example 5

### Differing Responses of Anti-GM1 mAbs DG1 and DG2 to Complexes of Gangliosides Containing GM1

Anti-GM1 mAbs DG1 and DG2 were applied to PVDF membranes or to ELISA plates at a concentration of 1 mg/ml. Using ELISA, DG1 (left panel) binds to GM1 alone, but with a weak signal for GM1/GD1a complex. DG2 (right panel) binds GM1 and is much less inhibited by the presence of GD1a (see FIG. 5A). Quantitative ELISA results from 4 independent experiments are shown in FIG. 5C. When using PVDF-glycoarrays, DG1 did not bind to complexes of GM1/GD1a, but bound to GM1 alone (see left hand panel of FIG. 5B), whereas DG2 bound to complexes of GM1/GD1a, as well as to GM1 alone (see right hand panel of FIG. 5B). Therefore, as can be seen in FIG. 5D, the inhibitory effect of GM1/GSL complexes on antibody binding is greater for DG1 than for DG2. These experiments show that the difference in the behaviour of the two antibodies with respect to binding of GM1 and GM1/GD1a complexes is more marked on PVDF as compared to ELISA.

Consistent with the results from the PVDF-based assays, DG1 failed to bind to nerve terminals in living tissue in which GM1/GSL complexes are thought to form (see FIG. 6G). The nerve terminals were identified by staining with bungarotoxin and detection using the cholera toxin was used to confirm expression of GM1 at these nerve terminals (see FIGS. 6 B, E, H and C). In contrast, DG2 was able to bind GM1 in the nerve terminals of living tissue (see FIG. 6D), confirming that the detection method is working in vivo. Thus, the PVDF-based method is able to identify that the DG1 antibody (which does not bind to GM1 in living tissue) is unable to bind to GM1 complexes, whereas ELISA is not able to discriminate as effectively. Therefore, the PVDF-based assay may be more representative of the in vivo situation, where GM1 is thought to exist as a complex, than the ELISA-based assay.

### Example 6

### A Comparison of mAb MOG26 and Siglec-E Reactivities on ELISA and PVDF-Glycoarrays

Identical preparations of MOG26 (FIGS. 7A and B) and siglec-E-Fc (FIGS. 7C and D) were investigated by ELISA and PVDF arrays. On ELISA, MOG26 reacts strongly with GM1/GD1a (see FIG. 7B), yet no signal is seen on PVDF (see FIG. 7A) for this complex (enclosed by circles) even when the antibody concentration is doubled form that used on ELISA and the exposure time is increased to 5 minutes. This reflects the situation seen in the live membrane of the GD3s^{-/-} mouse (in which GM1/GD1a complexes are expected to form) where this mAb also fails to bind (data not shown). Siglec-E binds GT1b/GM2 complex (enclosed by circles) in the PVDF system (see FIG. 7C), as well as showing reactivity towards GM2 (and to a lesser extent GT1b). On ELISA (see FIG. 7D), the signal is barely above baseline for GM2/GT1b, and absolutely undetectable for GM2 and GT1b alone. The graph is plotted on the same scale as for MOG26. These experiments demonstrate that reactivities can be seen on PVDF which are not replicated on ELISA, and vice versa.

### REFERENCES

1. Wenk M R. The emerging field of lipidomics. Nature Drug Discovery. 4, 594-610 (2005).
2. Regina, T. A. & Hakomori, S. I. Functional role of glycosphingolipids and gangliosides in control of cell adhesion, motility, and growth, through glycosynaptic microdomains. Biochim. Biophys. Acta 1780, 421-433 (2008).
3. Schiavo, G. & van der Goot, F. G. The bacterial toxin toolkit. Nat. Rev. Mol. Cell Biol. 2, 530-537 (2001).
4. Crocker, P. R., Paulson, J. C. & Varki, A. Siglecs and their roles in the immune system. Nat. Rev. Immunol. 7, 255-266 (2007).
5. Willison, H. J. The immunobiology of Guillain-Barre syndromes. J. Peripher. Nerv. Syst. 10, 94-112 (2005).
6. Kaida, K. et al. Ganglioside complexes as new target antigens in Guillain-Barre syndrome. Ann. Neurol. 56, 567-571 (2004).
7. Willison, H. J. et al. Inter-laboratory validation of an ELISA for the determination of serum anti-ganglioside antibodies. Eur. J. Neurol. 6, 71-77 (1999).
8. Kaida, K. et al. Anti-ganglioside complex antibodies associated with severe disability in GBS. Journal of Neuroimmunology. 182(1-2):212-8, (2007).
9. Willison, H. J. Ganglioside complexes: new autoantibody targets in Guillain-Barre syndromes. Nat. Clin. Pract. Neurol. 1, 2-3 (2005).
10. Chabraoui, F. et al. Dot-blot immunodetection of antibodies against GM1 and other gangliosides on PVDF-P membranes. J. Immunol. Methods 165, 225-230 (1993).
11. Kanter, J. L. et al. Lipid microarrays identify key mediators of autoimmune brain inflammation. Nat. Med. 12, 138-143 (2006).
12. Lloyd K O, Gordon C M, Thampoe I J, DiBenedetto C. 1992. Cell surface accessibility of individual gangliosides in malignant melanoma cells to antibodies is influenced by the total ganglioside composition of the cells. Cancer Res 52(18):4948-53.

1. A method comprising the steps of:
(i) providing a hydrophobic support displaying a lipid complex;
(ii) contacting the lipid complex with a sample; and
(iii) detecting binding of one or more components of the sample to the lipid complex.

2. A method according to claim 1 for detecting the presence of a lipid binding agent in the sample.

3. A method according to claim 1 wherein a sample which is known or suspected to contain an agent capable of binding to one or more lipid complexes is contacted with a plurality of different lipid complexes, said method optionally comprising detecting the presence of said agent in said sample.

4. A method according to claim 3 comprising the step of identifying the lipid complex or complexes to which binding occurs.

5. A method according to claim 4 wherein each of the plurality of lipid complexes is displayed at a defined, separate location on the support.

6. A method according to claim 5 comprising identifying the location on the support at which a positive binding reaction is obtained, and correlating that result with the identity of the complex displayed at that location.

7. A method according to claim 1 wherein the sample is a biological fluid selected from the group consisting of blood, serum, plasma, cerebrospinal fluid (CSF), saliva, mucous, or urine.

8. A method according to claim 1 wherein the sample comprises one or more cells or viruses, an extract of a cell or virus, or a component isolated therefrom.

9. A method according to claim 1 for use in the diagnosis of a disease characterised by the presence of a lipid binding agent.

10. A method according to claim 9 wherein the disease is an autoimmune.

11. A method according to claim 9 wherein the disease is caused by an infectious agent which produces a lipid binding agent.

12. A method according to claim 11 wherein the disease is cholera, tetanus, shigellosis, botulism or influenza.

13. A method according to claim 1 for use in determining whether a test compound in said sample is capable of binding to a lipid complex.

14. A method according to claim 13 wherein a plurality of test compounds are tested for their ability to bind to a lipid complex of choice.

15. A method according to claim 14 wherein the hydrophobic support carries the same lipid complex at a plurality of defined, separate locations, and wherein each sample comprising a test compound is contacted to an individual and distinct location on the support at which the lipid complex is present.

16. A method according to claim 15 comprising identifying the location at which a positive binding reaction is obtained, and correlating that result with the identity of the test compound in the sample applied to that location.

17. A method according to claim 16 for determining the amount of a lipid binding agent in a sample.

18. A method according to claim 17 for use in the diagnosis of a disease in which the levels of lipid binding agents are dysregulated.

19. A method according to claim 18 wherein the lipid binding agent is CD82.

20. A method according to claim 1 for determining whether a lipid binding agent binds to a particular lipid complex, wherein the sample contains a known lipid binding agent; and wherein the method comprises the step of detecting binding of said lipid binding agent to the lipid complex.

21. A method according to claim 20 comprising contacting the lipid binding agent with a plurality of lipid complexes in order to determine which complex or complexes are bound by the agent.

22. A method according to claim 21 wherein each of the plurality of different lipid complexes is displayed at a defined, separate location on the support.

23. A method according to claim 22 comprising identifying the location at which a positive binding reaction is obtained, and correlating that result with the identity of the complex displayed at that location.

24. A method of detecting the presence of a lipid complex in a sample, the method comprising the steps of:
(i) displaying the sample on a hydrophobic support;
(ii) contacting the sample with a known lipid binding agent; and
(iii) detecting binding of said lipid binding agent to the sample.

25. A method according to claim 24 wherein the sample comprises one or more cells, viruses, an extract of a cell or virus, or a component isolated therefrom.

26. A method according to claim 24 wherein the sample is, or is derived from, a tissue sample from an individual known or suspected to have a particular disease or to be infected with a particular pathogen.

27. A method according to claim 26 wherein a single sample is tested for reactivity with a plurality of known lipid binding agents.

28. A method according to claim 27 wherein the hydrophobic support carries a plurality of samples at defined separate locations.

29. A method according to claim 28 wherein the hydrophobic support is made from a material which has an advancing contact angle with respect to water selected from the group consisting of greater than 60°, greater than 65°, greater than 70°, greater than 75°, greater than 80°, greater than 85°, greater than 90°, greater than 95°, greater than 100°, greater than 105°, greater than 110°, and greater than 115°.

30. A method according to claim 28 wherein the hydrophobic support is made from polyvinylidene fluoride (PVDF), polytetrafluoroethylene (PTFE), polypropylene, polyethersulphate, polyetherimide (PEI), polyurethane, nylon, cellulose, nitrocellulose or silica.

31. A method according to claim 24 wherein a component of the lipid complex is selected from group consisting of a fatty acyl, a acid, glycerolipid, glycerophospholipid, sphingolipid, sterol or prenol.

32. A method according to claim 24 wherein one or more of the lipids is cholesterol, sphingomyelin, ceramide or digalactosyl diglyceride.

33. A method according to claim 24 wherein one or more of the lipids is a glycolipid.

34. A method according to claim 24 wherein one or more of the lipids is a non-glycosylated lipid.

35. A method according to claim 24 wherein the lipid complex comprises one or more glycolipids and/or one or more glycerophospholipids.

36. (Currently amended A method according to claim 24 wherein the lipid complex is a heterodimer or a homodimers.

37. A method according to claim 1 wherein the lipid complex contains three, four, five, six or more lipids.

38. A method according to claim 37 wherein the lipid complex is a complex of sulphatide, monosialoganglioside, cholesterol and phosphatidylethanolamine.

39. A method according to claim 36 wherein the lipid complex is a glycolipid complex and is a heterodimer of any two of the following gangliosides GM1, GM2, GM3, GDIa, GDIb, GD3, GTIa, GTIb, GDIb, GQIb and asialo-GM1 GM2/GT1b and GM1/GD1a.

40. A method according to claim 1 wherein the support carries every possible homodimeric and heterodimeric combination of a set of monomeric lipids, optionally wherein the set comprises at least 5, at least 10, or at least 15 monomeric lipids.

41. A method according to claim 40 wherein the same lipid complex is carried at a plurality of locations on the support.

42. A method according to claim 41 wherein the support comprises at least 100, at least 200, at least 500 or at least 1000 distinct locations each carrying a lipid complex.

43. A method according to claim 42 wherein the support comprises at least 10, at least 20, at least 50, at least 100, at least 200, at least 300, at least 400, or at least 500 distinct locations or lipid complexes per square centimetre.

44. A method according to claim 43 wherein individual locations or complexes are separated from adjacent locations or complexes by a barrier which acts to reduce or prevent fluid flow between locations.

45. A method according to claim 44 wherein the barrier is a hydrophobic barrier which resists fluid flow between adjacent locations or a wall.

46. A method according to claim 1 wherein the lipid binding is detected with a molecule selected from the group consisting of an antibody, a monoclonal antibody, serum immunoglobulin, a lectin, a siglec, a siglec-Fc fusion protein, a bacterial toxin, a cholera toxin, a tetanus toxin, a small molecule of 500 Da or less which possesses or is suspected to possess the capacity to bind lipid complexes.

47. A hydrophobic support displaying a lipid complex, or a plurality of lipid complexes at distinct defined locations.

48. A method of preparing a hydrophobic support according to claim 47, the method comprising the step of applying the lipid complex to the hydrophobic support.

49. A method according to claim 24 wherein the individual components of the complex are mixed together to allow interaction before they are applied to the support.

50. A kit for detecting binding of a lipid binding agent to a lipid complex, the kit comprising a hydrophobic support displaying a lipid complex according to claim 47.

51. The method as claimed in claim 10 wherein said autoimmune disease is selected from the group consisting of Guillain Barre syndrome (GBS) and multiple sclerosis.
